(19) United States
(12) Patent Application Publication
(10) Pub. No.: US 2016/0045719 A1
(43) Pub. Date: Feb. 18, 2016

(54) SHAFT SYSTEM FOR BALLOON DILATION

(71) Applicant: Acclarent, Inc., Menlo Park, CA (US)

(72) Inventors: Hung V. Ha, San Jose, CA (US); Ketan P. Muni, San Jose, CA (US)

(21) Appl. No.: 14/460,507

(22) Filed: Aug. 15, 2014

(51) Int. Cl. A61M 29/02; A61M 25/00

(52) U.S. Cl. CPC A61M 29/02; A61M 25/0026; A61M 25/0021; A61M 25/0023; A61B 17/24

(57) ABSTRACT

A multi-lumen catheter assembly comprises an inner shaft, an outer shaft and one or more features. The inner shaft comprises an inner surface and an outer surface. The inner surface defines a first lumen. The outer shaft is coaxially disposed about the inner shaft and is deformable inwardly toward the outer surface of the inner shaft. The outer shaft comprises an inner surface and an outer surface. The outer shaft is radially space from the inner shaft such that the inner surface of the outer shaft and the outer surface of the inner shaft together define a second lumen. The one or more features are configured to maintain patency through the second lumen as the outer shaft deforms inwardly toward the outer surface of the inner shaft.

### BACKGROUND

In some instances, it may be desirable to dilate an anatomical passageway in a patient. This may include dilation of ostia of paranasal sinuses, dilation of a patient's airway (e.g., to treat a stenosis within the larynx), dilation of the nasal cavity, dilation of the Eustachian tube, dilation of other passageways within the ear, nose, or throat, dilation of blood vessels, dilation of the urethra, etc. One method of dilating anatomical passageways includes using a guide wire and catheter to position an inflatable balloon within the anatomical passageway, then inflating the balloon with a fluid (e.g., saline) to dilate the anatomical passageway.

Airway stenosis (or “airway narrowing”) is a medical condition that occurs when some portion of a patient's airway becomes narrowed or constricted, thus making breathing difficult. A stenosis may occur in any part of the airway including the larynx, trachea, bronchi, or a combination of any of the above mentioned regions. Both adults and children may develop a stenosis. In some instances, a stenosis is caused by intubation, which is when a tube is placed in the airway for ventilation/breathing assistance in a patent who cannot breathe. Intubation for prolonged periods of time may traumatize the airway, causing scar tissue formation that forms the stenosis.

Therapies for treating an airway stenosis range from endoscopic treatments, such as dilation and laser resection, to open procedures, such as laryngotracheal reconstruction. In one technique, a series of rigid dilators of increasing diameter are pushed down the airway, gradually expanding the constriction but also applying shear forces to the airway. Balloon catheters may also be used to perform dilation of an airway or other anatomical passageway. For instance, the expandable balloon may be positioned within a stenosis in an airway (e.g., larynx, trachea, bronchi, etc.) and then be inflated, to thereby dilate the airway and increase airflow. The dilated airway may then allow for improved breathing. An example of a system that may be used to perform such procedures is described in U.S. Pub. No. 2010/0168511, entitled “System and Method for Dilating an Airway Stenosis,” published Jul. 1, 2010; and in U.S. patent application Ser. No. 13/795,791, entitled “Airway Dilation Shaft with Staggered Adjacent Internal Lumens,” filed on Mar. 12, 2013, the disclosures of which are incorporated by reference herein.

While several airway dilation systems have been made and used, it is believed that no one prior to the inventor(s) has made or used the invention described in the appended claims.

### BRIEF DESCRIPTION OF THE DRAWINGS

While the specification concludes with claims that particularly point out and distinctly claim this technology, it is believed this technology will be better understood from the following description of certain examples taken in conjunction with the accompanying drawings, in which like reference numerals identify the same elements and in which:

FIG. 1 depicts a side view of an exemplary system for dilating a stenosis in the airway, including a balloon catheter and a stylet;

FIG. 2 depicts a side view of the stylet of FIG. 1;

FIG. 3A depicts a cross-sectional end view of the system of FIG. 1 with the cross section taken along line 3-3 of FIG. 1

FIG. 3B depicts a cross-sectional end view of the system of FIG. 1, with the cross section taken along line 3-3 of FIG. 1, showing the balloon catheter collapsed against the stylet;

FIG. 4A depicts a cross sectional view of the system of FIG. 1 being introduced into an airway, with the balloon positioned at a stenosis in a collapsed state;

FIG. 4B depicts a cross sectional view of the system of FIG. 4A, with the balloon inflated to a dilated state;

FIG. 5A depicts a cross-sectional end view of an exemplary alternative balloon catheter for use in the system of FIG. 1;

FIG. 5B depicts a cross-sectional end view the balloon catheter of FIG. 5A with an outer shaft collapsed against an inner shaft;

FIG. 6A depicts a cross-sectional end view of another exemplary alternative balloon catheter for use in the system of FIG. 1;

FIG. 6B depicts a cross-sectional end view of the balloon catheter of FIG. 6A with an outer shaft collapsed against an inner shaft;

FIG. 7A depicts a cross-sectional end view of yet another exemplary alternative balloon catheter for use in the system of FIG. 1;

FIG. 7B depicts a cross-sectional end view of the balloon catheter of FIG. 7A with an outer shaft collapsed against an inner shaft;

FIG. 8A depicts a cross-sectional end view of still another exemplary alternative balloon catheter for use in the system of FIG. 1;

FIG. 8B depicts a cross-sectional end view of the balloon catheter of FIG. 8A with an outer shaft collapsed against an inner shaft;

FIG. 9A depicts a cross-sectional end view of still another exemplary alternative balloon catheter for use in the system of FIG. 1;

FIG. 9B depicts a cross-sectional end view of the balloon catheter of FIG. 9A with an outer shaft collapsed against an inner shaft;

FIG. 10 depicts a cross-sectional end view of an exemplary alternative multi-lumen balloon catheter for use in the system of FIG. 1;

FIG. 11 depicts a cross-sectional end view of another exemplary alternative multi-lumen balloon catheter for use in the system of FIG. 1; and

FIG. 12 depicts a cross-sectional end view of yet another exemplary alternative multi-lumen balloon catheter for use in the system of FIG. 1.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Overview of Exemplary Balloon Dilation Catheter System

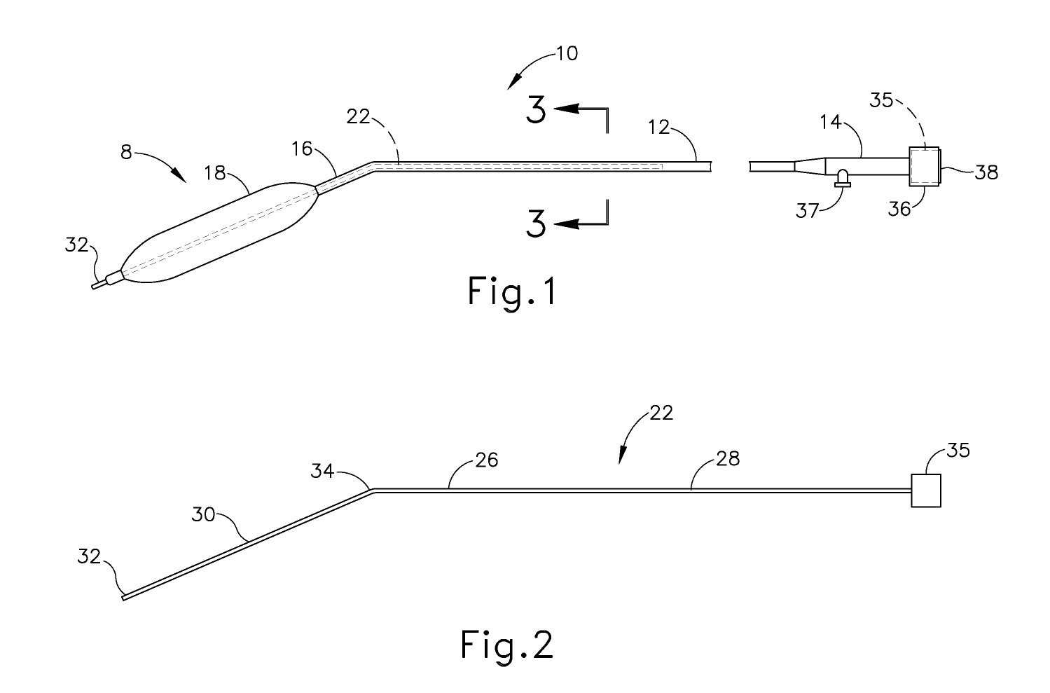

FIG. 1 shows an exemplary dilation catheter system (8), which may be used to dilate a stenosis in an airway; or to dilate some other anatomical passageway (e.g., within the ear, nose, throat, cardiovascular system, etc.). At least part of system (8) may be constructed and operable in accordance with at least some of the teachings of U.S. Pub. No. 2010/0168511, the disclosure of which is incorporated by reference herein. It should be understood that dilation catheter system (8) may be used to dilate either a naturally occurring passageway in a patient or a surgically created passageway in a patient.

Dilation catheter system (8) of this example comprises a balloon catheter (10) and a stylet (22). Balloon catheter (10) comprises a shaft assembly (12) positioned between a hub (14) and a balloon (18). Balloon (18) is coupled to a distal end of shaft assembly (12) and is configured to receive fluid through balloon catheter (10). Stylet (22) is slidably positioned through balloon catheter (10). In some versions, at least a portion of stylet (22) has a greater stiffness than at least a portion of balloon catheter (10), such that when stylet (22) is bent and inserted within balloon catheter (10), balloon catheter (10) at least partially conforms to the shape of stylet (22). In a dilation procedure, stylet (22) is used to advance balloon catheter (10) within an airway or targeted anatomical passageway (e.g., at a stenosis site). Balloon (18) may then be actuated to an expanded state to open or dilate the targeted anatomical passageway. Balloon (18) may then be actuated back to a collapsed state such that balloon (18) is deflated. This process may be repeated to dilate several anatomical passageways.

A. Exemplary Balloon Catheter

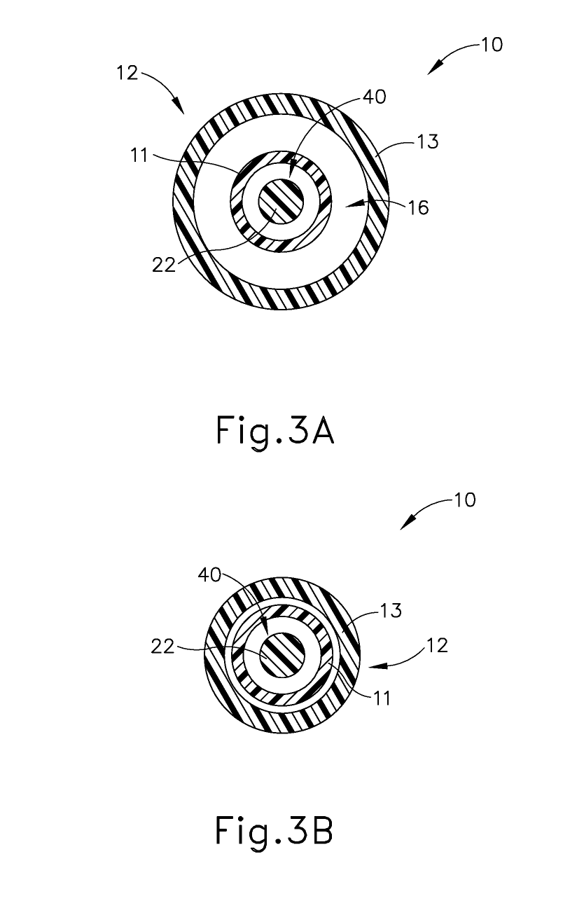

As shown in FIGS. 1 and 3A-3B, balloon catheter (10) of the present example comprises a catheter shaft assembly (12) and an inflatable balloon (18). Shaft assembly comprises a pair of coaxially aligned hollow shafts (11, 13). In particular, an inner shaft (11) extends through an outer shaft (13), and is radially spaced inwardly within outer shaft (13). The outer diameter of inner shaft (11) is sized smaller than the inner diameter of outer shaft (13). Accordingly, the relative sizing between shafts (11, 13) defines an inflation lumen (16) therebetween. Inflation lumen (16) provides a pathway for fluid to travel between inflation port (37) and balloon (18) such that balloon (18) may be selectively inflated and deflated. In particular, the proximal end of inflatable balloon (18) is attached to the distal end of outer shaft (13) via adhesive or other attachment means. The distal end of inflatable balloon (18) is attached to the distal end of inner shaft (11) via adhesive or other attachment means. Thus, the interior of inflatable balloon (18) is in fluid communication with inflation lumen (16), with the attachment regions of inflatable balloon (18) providing a fluid tight seal against shafts (11, 13). A hub (14) is coupled to a proximal end of shaft assembly (12) and comprises a stylet port (38) and an inflation port (37). Stylet (22) is insertable through stylet port (38) and further into an inner stylet lumen (40) of inner shaft (11). Inflation port (37) is in fluid communication with inflation lumen (16). In the present example, stylet lumen (40) is fluidly isolated relative to inflation lumen (16); and stylet port (38) is fluidly isolated relative to inflation port (37). Fluid (e.g., saline, etc.) may therefore be introduced through inflation lumen (16) via inflation port (37) to inflate balloon (18).

Balloon catheter (10) may have any number of suitable sizes, shapes and configurations. For example, balloon (18) may have different lengths and diameters in different embodiments, to accommodate different patient anatomies. The overall catheter (10) length and diameter may also vary. For example, the overall length of balloon catheter (10) (i.e., from the proximal end of hub (14) to the distal end of catheter shaft assembly (12)) is about 35-70 cm, such as less than or equal to about 50 cm, or about 45 cm.+−0.5 cm. Catheter (10) may be handled and manipulated with one hand. The working length of balloon (18) in FIG. 1 is about 40 mm +/−0.2 mm. By “working length” it is meant the length between the two tapered portions of balloon (18). In some versions, the working length of balloon (18) may range from between about 10 mm and about 60 mm such as about 16-45 mm. The outer diameter of the fully inflated working length of balloon (18) may also vary. In the present example, balloon (18) has an inflated diameter of about 14.1 mm +/−0.5 mm. In some versions, balloon (18) diameter may range from about 3 mm to about 24 mm, such as about 5-15 mm. A combination of balloon diameters and lengths may be provided, such that a physician may choose an appropriate size for an adult or pediatric patient. In one example, the following balloon diameters and lengths may be provided: 5 mm by 24 mm; 7 mm by 24 mm; 10 mm by 40 mm; and 14 mm by 40 mm. Of course, any of a number of other combinations of sizes of balloons (18) may be provided.

Any suitable material may be used to form balloon (18). Balloon (18) may be compliant, semi-compliant or non-compliant. Balloon (18) may be made of nylon, some other polymer, such as PTFE, and/or any other suitable material(s). In some versions, balloon (18) is formed of an elastic/extensible material that is resiliently biased to assume a shrunken, non-inflated configuration, such that the material forming balloon (18) is under increased tension when balloon (18) is in a non-deflated state. In some other versions, balloon (18) is formed of a material that is flexible yet substantially inelastic/non-extensible, such that the material forming balloon does not provide a significant resilient bias. In other words, balloon (18) does not stretch in response to increased fluid pressure inside balloon (18), even though the effective outer diameter of balloon (18) increases in response to increased fluid pressure. Such inelastic versions of balloon (18) may nevertheless be filled with fluid, with the fluid pressure being increased to provide an outwardly directed force via balloon (18), and this process may be referred to as “inflating.” When the pressure of fluid inside balloon (18) is reduced, this process may be referred to as “deflating,” even if the material forming balloon (18) does not elastically shrink, since balloon (18) may nevertheless flexibly collapse in response to reduced fluid pressure. Thus, it should be understood that the use of terms like “inflate,” “inflated,” “deflate,” and “deflated” does not necessarily mean that the material forming balloon (18) undergoes any elastic stretching or shrinking as the fluid pressure within balloon (18) changes.

In some versions, balloon (18) may include an outer slip-resistant surface, which may be formed by a textured surface or a coating. Such a surface may help prevent slipping of balloon (18) out of an airway structure during inflation and/or may facilitate re-wrapping balloon (18) by hand after deflation if balloon (18) is to be used for a second or subsequent dilation procedure. Examples of such balloons are provided in U.S. patent application Ser. No. 13/796,073, entitled “Features to Enhance Grip of Balloon within Airwary,” filed on Mar. 12, 2013, the disclosure of which is incorporated by reference herein.

Inner shaft (11) or outer shaft (13) may also be formed of any suitable material. It may be desirable to form shafts (11, 13) from material(s) selected so that shafts (11, 13) are unlikely to kink when bent, such as when bent by stylet (22) and/or a user. One such material, for example, is Pebax, although other polymers may be used. Additionally, inner shaft (11) or outer shaft (13) may be formed of the same or dissimilar materials. Inner shaft (11) or outer shaft (13) may also have any suitable color and may include one or more shaft markings. The shaft color and markings may be built into shafts (11, 13) by using a colored material or may be added by applying paint or another colorant. In some versions, shafts (11, 13) may have a dark color, such as black or dark blue, and one or more light colored markings may be applied over the dark shafts (11, 13). In some versions, the markings (not shown) may include direct visualization markings (viewed directly with the naked eye or an endoscope) and/or radiographic markings (viewed with a radiographic device such as intraoperative fluoroscopy). Any suitable combination, size and color of markings may be used. One example of shaft color and shaft markings, which could be used or modified for balloon catheter (10), is the Relieva Solo Pro Sinus Balloon Catheter, manufactured by Acclarent, Inc. of Menlo Park, Calif.

B. Exemplary Stylet

FIG. 2 shows stylet (22) in greater detail. Stylet (22) comprises a core member (26) with a proximal section (28) and a distal section (30). A coil (32) is disposed around at least part of distal section (30) of core member (26). A luer lock member (35) is coupled with a proximal end of core member (26) for coupling with a hub (14) on balloon catheter (10). In some versions, stylet (22) does not include a coil (32). Core member (26) and/or coil (32) may be formed of nitinol, stainless steel, or other biocompatible materials. Distal section (30) of stylet (22) includes a bend or curve (34) that is stiff enough to bend balloon catheter (10) during the placement of balloon catheter (10) within the airway of the patient. In some versions, stylet (22) may be provided in a generally straight configuration. Stylet (22) may be pre-formed to have a bend (34), or stylet (22) may be malleable, such that a user may bend stylet (22) and stylet (22) maintains the user-created bend. This malleability allows a user to adjust a bend angle according to the airway anatomy of a particular patient. Proximal section (28) of stylet (22) may be generally stiff, a distal section (30) may be generally malleable, and an extreme distal portion may be atraumatic and very flexible or even floppy. This variation in flexibility along the length of stylet (22) may be achieved by using different materials, such as stainless steel and nitinol. Alternatively, one material, such as stainless steel, may be used and the diameter of stylet (22) may be altered to achieve the variation in flexibility along the length of stylet (22).

Stylet (22) has an overall length approximately as long or slightly longer than balloon catheter (10). In some versions, stylet (22) includes an atraumatic, flexible distal tip portion that extends distally out of balloon catheter (10) when stylet (22) is fully disposed within catheter (10). This tip portion may be, for example, between about 0.25 cm to about 8 cm (e.g., about 1-5 cm) in length; and may facilitate the ability of a user to advance system (8) through a patient's airway atraumatically. The overall length of stylet (22) may vary from about 30 cm to about 80 cm, such as from about 45 cm to about 60 cm. Of the overall length, a flexible distal portion of stylet (22) may be from about 5-20 cm, such as from about 10-15 cm. Bend (34) may have any suitable angle, such as from greater than 0 degrees to about 20 degrees. The diameter of stylet (22) may be less than about 1.3 mm, such as 0.9 mm or less. The diameter may decrease distally to about 0.13 mm+/−0.013 mm. Of course, the foregoing dimensions are mere examples. Any other suitable dimensions may be used.

Stylet (22) may be attached to balloon catheter (10), or stylet (22) may be removably connected to balloon catheter (10). Stylet (22) comprises a luer lock member (35) with threads on proximal section (28) that screw into opposing threads disposed on a luer (36) of balloon catheter (10). In some versions, balloon catheter (10) may include a locking mechanism (not shown) to lock stylet (22) in position within catheter (10). The locking mechanism can be any mechanical device, including a lever, a ball and pin, a luer, etc. All or part of distal section (30) of stylet (22) may extend out of the distal end of catheter (10). Stylet (22) may be locked to balloon catheter (10) at different positions or lengths so the distal end of stylet (22) extends out of or is positioned within balloon catheter (10) at different lengths. The length, diameter(s) and stiffness characteristics of stylet (22) may be varied in different embodiments to confer different performance characteristics to the overall system (8).

Use of stylet (22) to insert balloon catheter (10) helps to guide the distal end of balloon catheter (10) through the airway of the patient and to the stenotic region. Stylet (22) provides increased steerability during advancement of balloon catheter (10). Torquability of balloon catheter (10) is also increased when using stylet (22). In some versions, luer lock member (35) of stylet (22) and luer (36) of balloon catheter (10) mate together, so that stylet (22) and balloon catheter (10) may be rotated together and thus steered into a constricted portion of an airway.

In some versions, stylet (22) may have a light emitting portion, such as a light emitting distal end or tip. For example, stylet (22) may include one or more light fibers to transmit light from a light source attached to the proximal end of stylet (22) to its distal end. Light from a light emitting stylet (22) may be used to help a user visualize a patient's airway from the inside using a scope and/or in some cases from the outside via transillumination through the patient's skin. A light emitting guidewire device that may be used or modified to achieve such an illuminating stylet (22) is the Relieva Luma™ Sinus Illumination Guidewire/System, manufactured by Acclarent, Inc. of Menlo Park, Calif. Such an illuminating stylet (22) may have any of the features described above with the additional feature of light emitting capability.

As can be seen in FIG. 3A, outer shaft (13), inner shaft (11), and stylet (22) are approximately coaxial with each other when stylet (22) is inserted into balloon catheter (10). While stylet (22) is inserted in stylet lumen (40) in the present example, it should be understood that a guidewire, optical fiber(s), endoscope, and/or various other kinds of structures may be inserted in stylet lumen (40), depending on the inner diameter size of stylet lumen (40). It should also be understood that stylet lumen (40) is entirely optional and may be omitted in some examples.

C. Exemplary Method of Use of the System

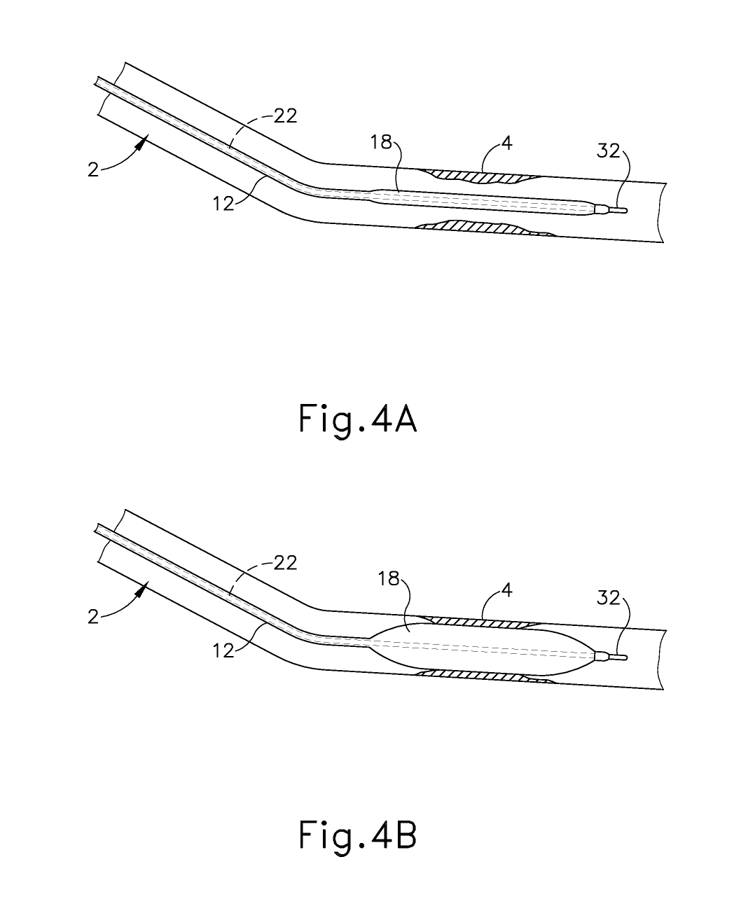

FIGS. 4A and 4B show a method for dilating an stenotic region (4) in an airway (2), such as in a case of subglottic stenosis. Dilation system (8) is introduced through the mouth and into the airway of the patient. Optionally, a bronchoscope (not shown) or other scope device may be used to visualize the positioning of dilation system (8). Dilation system (8) may be bent either by the user or by the manufacturer of system (8). For example, stylet (22) may be bent and then inserted into balloon catheter (10), while in other cases stylet (22) and balloon catheter (10) may be bent together, with stylet (22) already residing in catheter (10). The support of stylet (22) and the bend in the overall system (8) may help a physician navigate system (8) through the patient's airway to position balloon (18) within at least a portion of stenotic region (4). As shown in FIG. 4A, inflatable balloon (18) of the catheter (10) is in an unexpanded configuration during advancement and placement of balloon catheter (10). As shown in FIG. 4B, once balloon (18) is positioned within stenotic region (4) of the airway (2), inflatable balloon (18) is inflated to dilate stenotic region (4). Balloon (18) is then deflated to enable removal from airway (2). By way of example only, balloon (18) may be deflated by actively drawing the fluid from balloon (18); by venting the fluid in balloon (18), allowing the inward pressure imposed by airway (2) to drive fluid from balloon (18); or in any other suitable fashion as will be apparent to those of ordinary skill in the art in view of the teachings herein.

In some versions, stylet (22) remains in balloon catheter (10) during inflation of balloon (18). Maintaining stylet (22) in catheter (10) during inflation may give catheter (10) added column strength and help maintain the position of balloon (18) within stenotic region (4), thus avoiding slipping. In some versions, stylet (22) is removed from balloon catheter (10) before inflating. Stylet (22) may be removed from balloon catheter (10) after balloon catheter (10) is properly positioned within airway (2) of the patient, or stylet (22) can be removed after stenosis (4) has been dilated but before removing balloon catheter (10) from the patient.

Inflatable balloon (18) may be inflated more than once to dilate stenotic region (4) of airway (2). The physician inflates inflatable balloon (18) to a desired pressure during each dilation of stenosis (4). Proper dilation of stenotic region (4) can be confirmed by visualizing the region with the bronchoscope/endoscope.

II. Exemplary Alternative Balloon Catheters

In some instances, as can be seen in FIG. 3B, an operator may misuse balloon catheter (10) by attempting to extract balloon catheter (10) from a patient while balloon (18) is still inflated. Such premature removal may cause the inflated balloon (18) to catch on anatomy of a patient (e.g., stenosis (4)), which may further cause outer shaft (13) to bend, collapse, or neck inwardly against inner shaft (11), thus blocking or sealing off inflation lumen (16). Such blocking or sealing of inflation lumen (16) may substantially restrict the deflation rate of balloon (18) or even completely prevent deflation of balloon (18). When balloon (18) will not deflate quickly enough (or not deflate at all), due to outer shaft (13) blocking or sealing inflation lumen (16), further attempts to extract balloon catheter (10) may lead to tearing of balloon catheter (10). In some instances, this may ultimately lead to balloon (18) breaking free from shaft assembly (12) and becoming lodged in the patient's airway.

In view of the foregoing, it may be desirable to utilize a dilation catheter system (8) with a balloon catheter having multiple coaxial shafts or differing shaft configurations. The merely illustrative examples described below may provide enhanced resistance to kinking, enhanced collapse strength, and/or other effects that may reduce or eliminate risks that may otherwise be triggered by operator error. In some versions, these enhanced effects are provided by incorporating additional elements into the structure of a balloon catheter to maintain separation between coaxial shafts. This separation between coaxial shafts provides a lumen through which fluid may be communicated between balloon (18) and a proximal end of the coaxial shaft assembly. By maintaining at least some degree of separation between coaxial shafts, the examples described below maintain patency through the lumen that is defined between the coaxial shafts. In the examples described herein, the term “maintain patency” should be understood to mean that the lumen provides enough patency to enable balloon (18) to be deflated within a clinically acceptable time frame, even when forces are exerted on the outer shaft that urge the outer shaft inwardly toward the inner shaft. A “clinically acceptable time frame” is one in which balloon (18) deflates quickly enough for balloon (18) to be pulled from the patient's airway without tearing or otherwise rupturing balloon catheter (10) and/or the interface of balloon (18) and some other portion of balloon catheter (10).

In some other versions, patency is maintained by mechanically separating the axes of lumens within a balloon catheter having a single shaft. In other words, creating mechanical separation between areas of fluid communication may provide enhanced effects not provided by conventional balloon catheters (10). Various other effects that may be provided by incorporating additional elements into the balloon catheter or separating lumen axes will be apparent to those of ordinary skill in the art in view of the teachings herein.

It should be understood that the exemplary catheter assemblies described below provide a relatively low profile and also substantial flexibility. The exemplary catheter assemblies described below are therefore suitable for use in a patient's airway, to dilate a stenosis and/or for other purposes. It should also be understood that the following examples are merely illustrative, and that other variations will also be apparent to those of ordinary skill in the art in view of the teachings herein.

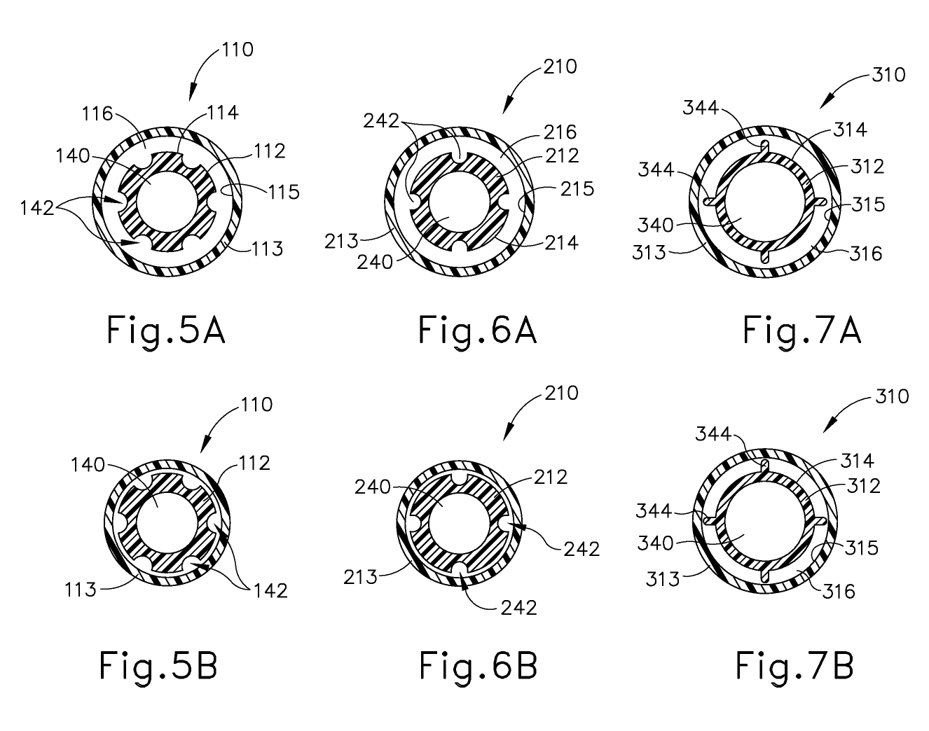

FIG. 5A shows a cross-section of an exemplary alternative balloon catheter (110) that may be used as an alternative to balloon catheter (10) in the dilation catheter system depicted in FIG. 1. Unless otherwise noted below, balloon catheter (110) has substantially the same construction as balloon catheter (10). In particular, balloon catheter (110) comprises an inner shaft (112) oriented coaxially with an outer shaft (113). An exterior surface (114) of inner shaft (112) together with an interior surface (115) of outer shaft defines a lumen (116) between inner shaft (112) and outer shaft (113). When balloon catheter (110) is in the configuration shown in FIG. 5A, lumen (116) may communicate fluid to a balloon (not shown), like balloon (18), on the distal end of balloon catheter (110).

As can be seen, inner shaft (112) is configured with an inner lumen (140) running through its center. Although only a single lumen (140) is shown, it should be understood that other examples may include multiple lumens or inner lumen (140) may be omitted entirely. In the present example, inner lumen (140) may be used as a passage for stylet (22). Of course, in other versions, stylet (22) may be omitted and internal lumen (140) may be used for any other suitable purpose as will be apparent to those of ordinary skill in the art in view of the teachings herein.

Inner shaft (112) also includes a plurality of angularly spaced recesses (142) positioned on exterior surface (114) of inner shaft (112). Recesses (142) are shown as being semicircular in shape, with each individual recess (142) having a substantially similar shape. Additionally, each individual recess (142) is shown as being substantially evenly spaced apart from the next recess (142). Although recesses (142) are shown as being six semicircular recesses (142) spaced evenly about an exterior of inner shaft (112), it should be understood that any suitable configuration may be used. Indeed, recesses (142) may be square, triangular, ovular, or any other suitable shape. Similarly, recesses (142) may be larger or smaller than depicted and may be more or less in quantity that the six depicted. Recesses (142) may further extend along the full length of inner shaft (112) or part of inner shaft (112), each recess (142) taking the form of a longitudinally extending groove. Of course, any other configuration, such as differently spaced recesses (142) may be used as will be apparent to those of ordinary skill in the art in view of the teachings herein.

FIG. 5B shows balloon catheter (110) with outer shaft (113) in a collapsed state which may occur during misuse. In such a state all or at least a portion of, outer shaft (113) may collapse inwardly upon the exterior of inner shaft (112). When such a condition occurs, FIG. 5B shows that each recess (142) may provide a passage for fluid communication even if the regions of lumen (116) adjacent to recesses (142) are blocked. Accordingly, fluid can be evacuated from the balloon via lumen (116) at recesses (142) even when outer shaft (113) fully or partially collapses relative to inner shaft (112). In other words, balloon catheter (110) maintains patency through lumen (116) via recesses (142) when outer shaft (113) is in a collapsed state.

FIG. 6A shows a cross-section of another exemplary alternative balloon catheter (210) similar to balloon catheters (10, 110). Unless otherwise noted below, balloon catheter (210) has substantially the same construction as balloon catheters (10, 110). Like with balloon catheter (110), balloon catheter (210) comprises an inner shaft (212) oriented coaxially within an outer shaft (213). An exterior surface (214) of inner shaft (212) together with an interior surface (215) of outer shaft defines a lumen (216) between inner shaft (212) and outer shaft (213). When balloon catheter (210) is in the configuration shown in FIG. 6A, lumen (216) may communicate fluid to a balloon (not shown), like balloon (18) on the distal end of balloon catheter (210). Balloon catheter (210) also has a single inner lumen (240) extending through the center of inner shaft (212). Similarly to inner shaft (112), inner shaft (212) may utilize multiple lumens (240) or no lumen at all. Likewise, inner lumen (240) may be used to accommodate stylet (22) or any other suitable device.

Inner shaft (212) also includes a plurality of angularly spaced recesses (242) positioned on exterior surface (214) of inner shaft (212). Like with recesses (142), recesses (242) are shown as being semicircular in shape, with each individual recess (142) having a substantially similar shape. Also like recesses (142), each individual recess (242) is shown as being substantially evenly spaced apart from the next recess (242). However, unlike recesses (142), the present example includes four recesses (242) (as opposed to six recesses (142)). Additionally, FIG. 6A depicts some variation in size and shape of each recess (242) as compared to recesses (142). Although recesses (242) are shown as being four semicircular recesses (242) spaced evenly about an exterior of inner shaft (212), it should be understood that any suitable configuration may be used. Indeed, recesses (242) may be square, triangular, ovular, or any other suitable shape. Similarly, recesses (242) may be larger or smaller than depicted and may be more or less in quantity that the four depicted. Recesses (242) may further extend along all or part of the full length of inner shaft (212), each recess (242) taking the form of a longitudinally extending groove. Of course, any other configuration, such as differently spaced recesses (242) may be used as will be apparent to those of ordinary skill in the art in view of the teachings herein.

Similar to FIG. 5B, discussed above, FIG. 6B shows balloon catheter (210) with outer shaft (213) in a collapsed state which may occur during misuse. In such a state all or at least a portion of, outer shaft (213) may collapse inwardly upon the exterior of inner shaft (212). When such a condition occurs, FIG. 6B shows that each recess (242) may provide a passage for fluid communication. Accordingly, fluid can be evacuated from the balloon via (216) lumen at recesses (242) even when outer shaft (213) fully or partially collapses relative to inner shaft (212). In other words, balloon catheter (210) maintains patency through lumen (216) via recesses (242) when outer shaft (213) is in a collapsed state.

FIG. 7A shows a cross-section of yet another exemplary alternative balloon catheter (310) similar to balloon catheters (10, 110, 210). Unless otherwise noted below, balloon catheter (310) has substantially the same construction as balloon catheters (10, 110, 210) Like with balloon catheters (110, 210), balloon catheter (310) comprises an inner shaft (312) oriented coaxially within an outer shaft (313). An exterior surface (314) of inner shaft (312) together with an interior surface (315) of outer shaft defines a lumen (316) between inner shaft (312) and outer shaft (313). When balloon catheter (310) is in the configuration shown in FIG. 7A, lumen (316) may communicate fluid to a balloon (not shown), like balloon (18), on the distal end of balloon catheter (310). Balloon catheter (310) has a single inner lumen (340) extending through the center of inner shaft (312). Similar to inner shaft (112), inner shaft (312) may utilize multiple lumens (340) or no lumen at all. Likewise, inner lumen (340) may be used to accommodate stylet (22) or any other suitable device as will be apparent to those of ordinary skill in the art in view of the teachings herein.

Unlike inner shafts (112, 212), discussed above, inner shaft (312) includes a plurality of angularly spaced protrusions (344). In particular, four protrusions (344) extend outwardly from the exterior surface (314) of inner shaft (312), each protrusion (344) having the shape of a rounded rectangle. Each individual protrusion (344) is shown as being substantially evenly spaced apart from the next protrusion (344). Although protrusions (344) are shown as being four semi-rectangular outwardly extending projections spaced evenly about an exterior of inner shaft (312), it should be understood that any suitable configuration may be used. Indeed, protrusions (344) may be any suitable shape such as square, triangular, ovular, or etc. Similarly, protrusions (344) may be larger or smaller than depicted and may be more or less in quantity that the four depicted. Additionally, protrusions (344) may extend along all or part of the length of inner shaft (312). Of course, any other configuration, such as differently spaced recesses (344) may be used as will be apparent to those of ordinary skill in the art in view of the teachings herein.

Although protrusions (344) are different from recesses (142, 242), it can be seen in FIG. 7B that protrusions (344) function similarly to recesses (142, 242). In particular, FIG. 7B shows outer shaft (313) in a collapsed state, as similarly discussed above. When outer shaft (313) is in the collapsed state, protrusions (344) are operable to maintain at least some clearance (346) between inner shaft (312) and outer shaft (313) to maintain patency through lumen (316). Accordingly, protrusions (344) permit fluid to be evacuated from the balloon via lumen (316) even when outer shaft (313) is in the collapsed state. In other words, balloon catheter (310) maintains patency through lumen (316) due to spacing maintained by protrusions (344) when outer shaft (313) is in a collapsed state.

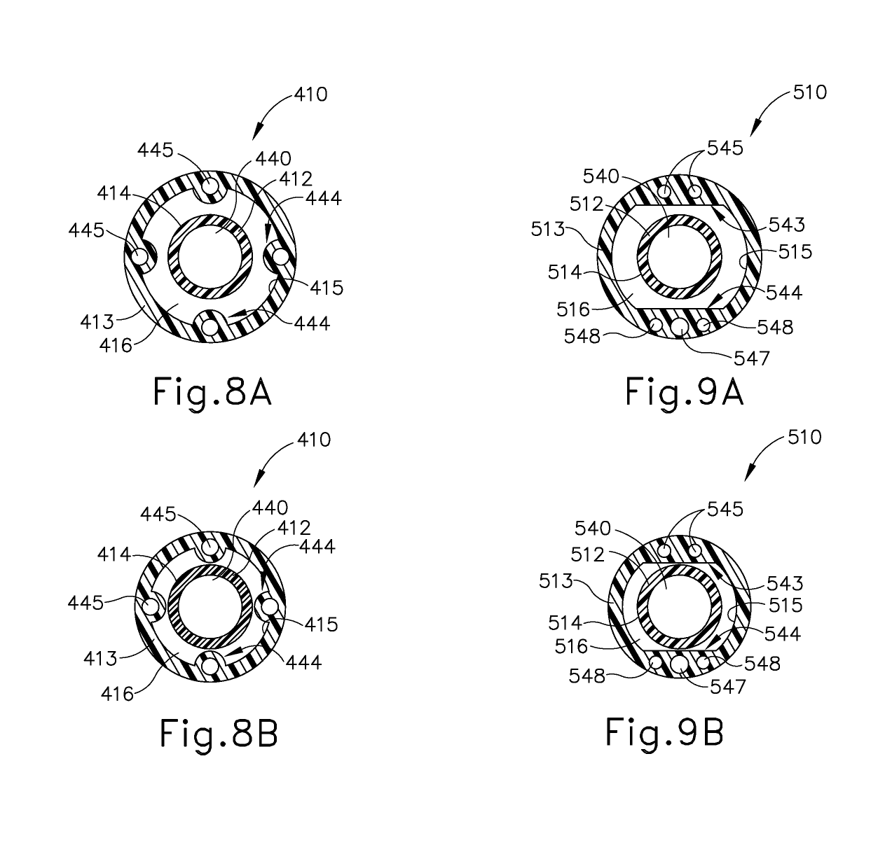

FIG. 8A shows a cross-sectional view of still another exemplary alternative balloon catheter (410) that may be used with the dilation catheter system (8) of FIG. 1. Unless otherwise noted below, balloon catheter (410) is substantially the same as balloon catheters (10, 110, 210, 310), having the same features and components. Like the other coaxial type balloon catheters (110, 210, 310), discussed above, balloon catheter (410) comprises an inner shaft (412) coaxially disposed within an outer shaft (413). An exterior surface (414) of inner shaft (412) together with an interior surface (415) of outer shaft defines a lumen (416) between inner shaft (412) and outer shaft (413). When balloon catheter (410) is in the configuration shown in FIG. 8A, lumen (416) may communicate fluid to a balloon (not shown), like balloon (18), on the distal end of balloon catheter (410).

Inner shaft (412) defines an inner lumen (440) that extends longitudinally though inner shaft (412). Similar to inner lumens (140, 240, 340) inner lumen (440) may consist of a single lumen, multiple lumens, or inner lumen (440) may be omitted entirely. Likewise, inner lumen (440) may be configured to receive stylet (22) or any other suitable device as will be apparent to those of ordinary skill in the art in view of the teachings herein.

Unlike other coaxial type balloon catheters (110, 210, 310) discussed above, balloon catheter (410) comprises four protrusions (444) extending inwardly from interior surface (415) of outer shaft (413) spaced equidistantly around the interior diameter of outer shaft (413). In particular, protrusions (444) are semi-circular in shape such that each protrusion (444) defines a lumen (445) centered within the semi-circular shape of each protrusion (444). Protrusions (444) may extend along the full length of outer shaft (413) or part of outer shaft (413). In other versions, each protrusion (444) may define more than one lumen (445). Yet in other versions, lumen (445) of each protrusion (444) may be omitted entirely. Similarly, the orientation of each protrusion (444) or the individual shape of each protrusion (444) may be varied. For instance, there may be more or less than four protrusions (444) and/or protrusions (444) may have separated from each other by varying distances. Additionally, protrusions (444) may have other suitable shapes such as triangular, square, rectangular, or a rounded variation of such shape. As will be discussed in greater detail below, lumens (445) of protrusions (444) may be used for communication of fluid. In other versions, lumens (445) may be used for communication of other components such as stylets, guide wires, push/pull rods, electrical wires, and/or etc.

Although protrusions (444) are different from recesses (142, 242) and protrusions (344), discussed above, it can be seen in FIG. 8B that protrusions (444) function similarly to recesses (142, 242) and protrusions (344). In particular, FIG. 8B shows outer shaft (413) in a collapsed state, as similarly discussed above. When outer shaft (413) is in the collapsed state, protrusions (444) are operable to maintain patency through lumen (416) between exterior surface (414) of inner shaft (412) and interior surface (415) of outer shaft (413). Accordingly, protrusions (444) permit fluid to be evacuated through balloon catheter (410) via lumen (416) near protrusions (444) even when outer shaft (413) is in the collapsed state. Moreover, lumen (445) of each protrusion (444) may provide an additional fluid pathway for evacuation of fluid. Thus, balloon catheter (410) maintains patency through lumen (416) and/or through lumens (445) when outer shaft (413) is in a collapsed state.

FIG. 9A shows a cross-sectional view of still another exemplary alternative balloon catheter (510) that may be used with the dilation catheter system (8) of FIG. 1. Unless otherwise noted below, balloon catheter (510) is substantially the same as balloon catheters (10, 110, 210, 310, 410), having the same features and components. Like the other coaxial type balloon catheters (110, 210, 310, 410), discussed above, balloon catheter (510) comprises an inner shaft (512) coaxially disposed within an outer shaft (513). An exterior surface (514) of inner shaft (512) together with an interior surface (515) of outer shaft defines a lumen (516) between inner shaft (512) and outer shaft (513). When balloon catheter (510) is in the configuration shown in FIG. 9A, lumen (516) may communicate fluid to a balloon (not shown), similar to balloon (18), on the distal end of balloon catheter (510).

Inner shaft (512) defines an inner lumen (540) that extends longitudinally though inner shaft (512). Similar to inner lumens (140, 240, 340, 440) inner lumen (540) may consist of a single lumen, multiple lumens, or inner lumen (540) may be omitted entirely. Likewise, inner lumen (540) may be configured to receive stylet (22) or any other suitable device as will be apparent to those of ordinary skill in the art in view of the teachings herein.

Unlike other coaxial type balloon catheters (110, 210, 310, 410), discussed above, balloon catheter (510) comprises two flat portions (543, 544) oriented on opposite sides of interior surface (515) of outer shaft (513). In particular, flat portions (543, 544) comprise a portion of outer shaft (513) where the wall of outer shaft (513) has a greater thickness relative to the rest of outer shaft (513). As can be seen in FIG. 9A, the increased wall thickness of flat portions (543, 544) provides a region for a plurality of lumens (545, 547, 548). In the present example, flat portion (543) has two equally sized lumens (545), while flat portion (544) has three lumens (547, 548) of varying sizes. As can be seen, lumen (545) are larger relative to lumens (548) and lumen (547) is larger than lumens (545, 548). In other versions, the particular number of lumens (545, 547, 548) of each flat portion (543, 544) may be varied. For instance, each flat portion (543, 544) may have two lumens (545) or each flat portion (543, 544) may have three lumens (547, 548). Yet in other versions, any suitable number or size of lumens (545, 547, 548) may be used as will be apparent to those of ordinary skill in the art in view of the teachings herein. Of course, as similarly described above, lumens (545, 547, 548) may be omitted entirely. As will be discussed in greater detail below, lumens (545, 547, 548) of flat portions (543, 544) may be used for communication of fluid. In other versions, lumens (545, 547, 548) may be used for communication of other components such as stylets, guide wires, push/pull rods, electrical wires, and/or etc.

Although flat portions (543, 544) are different from recesses (142, 242) and protrusions (344, 444), discussed above, it can be seen in FIG. 9B that flat portions (543, 544) function similarly to recesses (142, 242) and protrusions (344, 444). In particular, FIG. 9B shows outer shaft (513) in a collapsed state, as similarly discussed above. Outer shaft (513) collapses at the thinner walled regions, not at flat portions (543, 544), due to the respective difference in wall thicknesses. Accordingly, flat portions (543, 544) permit fluid to be evacuated through balloon catheter (510) via lumen (516) at flat portions (543, 544), even when outer shaft (513) is in the collapsed state. Moreover, lumens (545, 547, 548) of each protrusion (544) may provide an additional fluid pathway for communication of fluid to inflate or deflate the balloon. Thus, balloon catheter (510) maintains patency through lumen (516) and/or through lumens (545, 547, 548) when outer shaft (513) is in a collapsed state.

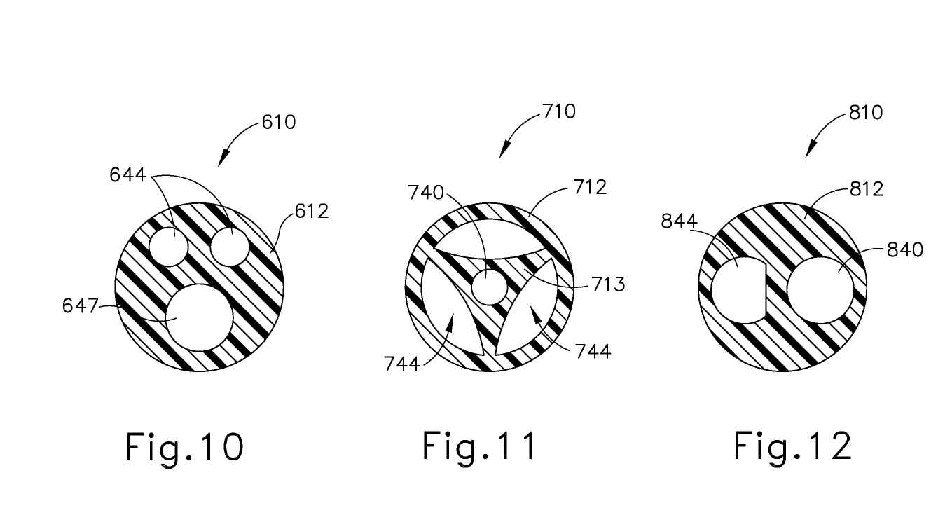

FIG. 10 shows a cross-section of an exemplary alternative single shaft balloon catheter (610) that may be used with the dilation catheter system (8) of FIG. 1. As can be seen, balloon catheter (610) comprises a single shaft (612) having a plurality of lumens (644, 647) extending longitudinally there through. Lumens (644, 647) consist of two similarly sized inflation/deflation lumens (644) that are smaller relative to a stylet lumen (647). In other versions, shaft (612) may comprise any suitable number of lumens (644, 647) having any suitable shape or size as will be apparent to those of ordinary skill in the art in view of the teachings herein. In the present example, lumens (644) are used for communication of fluid to inflate/deflate a balloon (not shown), which may be similar to balloon (18). Lumen (647) is used to receive a stylet (not shown), which may be similar to stylet (22). In other examples, lumens (647) may also be used for communication of fluid. In still other versions, lumens (644) may also be used for communication of other components dilation catheter system (8) such as stylets, guide wires, push/pull rods, electrical wires, and/or etc. If balloon catheter (610) is pulled while inflated, patency will be maintained through inflation/deflation lumens (644) due to the thickness of balloon catheter (610).

FIG. 11 shows a cross-section of another exemplary alternative single shaft balloon catheter (710) that may be used with the dilation catheter system (8) of FIG. 1. As can be seen, balloon catheter (710) comprises a single shaft (712) having integral three point stabilizing member (713). Stabilizing member (713) includes an inner lumen (740) that is centrally located within shaft (712). Additionally, stabilizing member (713) defines a plurality of generally ovular outer lumens (744) which occupy the space between stabilizing member (713) and shaft (712). Outer lumens (744) are of a substantially similar size and shape and are oriented at equal distances around the inside of shaft (712). In the present example, inner lumen (740) is sized and shaped to receive stylet (22) of dilation catheter system (8). However, in other versions inner lumen (740) may be configured to communicate fluids and/or other devices such as guide wires, push/pull rods, electrical wires or the like. Outer lumens (744) are configured to communicate fluid to a balloon (not shown), similar to balloon (18), such that the balloon may be inflated or deflated as described above. In particular, larger, discrete cross-sections of outer lumens (744) make them less prone to collapse when balloon catheter (710) is pulled while the balloon is inflated. In addition or in the alternative, if one outer lumen (744) collapses, at least one other outer lumen (744) may have patency. Like with inner lumen (740), outer lumens (744) may be configured to communicate devices such as stylets, guide wires, push/pull rods, electrical wires, and/or etc. in addition to, or in lieu of communicating fluid. Of course, any other suitable combination of lumen quantity, size, and/or shape may be used as will be apparent to those of ordinary skill in the art in view of the teachings herein.

FIG. 12 shows a cross-section of yet another exemplary alternative single shaft balloon catheter (810) that may be used with the dilation catheter system (8) of FIG. 1. Balloon catheter (810) comprises a single solid shaft (812) with a circular first lumen (840) and a D-shaped second lumen (844) extending longitudinally therethrough. First lumen (840) is shaped and sized to receive stylet (22) of dilation catheter system (8). Likewise, second lumen (844) is sized and shaped to communicate fluid to a balloon (not shown), similar to balloon (18), to inflate/deflate the balloon. Like with balloon catheter (610), the additional thickness of the sidewall of balloon catheter (810) maintains patency through second lumen (844) if balloon catheter (810) is pulled while the balloon is inflated. In other versions, both first lumen (840) and second lumen (844) may be used to receive stylet (22) or communicate fluid. Yet in other versions, the number, size, and shape of each lumen (840, 844) may be varied to receive other devices such as additional stylets, guide wires, push/pull rods, electrical wires, and the like. Of course, any other suitable combination of lumen quantity, size, and/or shape may be used as will be apparent to those of ordinary skill in the art in view of the teachings herein.

III. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A multi-lumen catheter assembly, comprising:
(a) an inner shaft, wherein the inner shaft comprises:
(i) an inner surface, wherein the inner surface defines a first lumen, and
(ii) an outer surface;
(b) an outer shaft coaxially disposed about the inner shaft, wherein the outer shaft is deformable inwardly toward the outer surface of the inner shaft, wherein the outer shaft comprises:
(i) an inner surface, and
(ii) an outer surface,
wherein the outer shaft is radially spaced from the inner shaft such that the inner surface of the outer shaft and the outer surface of the inner shaft together define a second lumen;
(c) an inflatable member in fluid communication with the second lumen; and
(d) one or more features configured to maintain patency through the second lumen as the outer shaft deforms inwardly toward the outer surface of the inner shaft.

2. The apparatus of claim 1, wherein the one or more features are positioned on the outer surface of the inner shaft.

3. The apparatus of claim 2, wherein the one or more features comprise a plurality recesses formed in the outer surface of the inner shaft.

4. The apparatus of claim 3, wherein the plurality of recesses comprise four recesses.

5. The apparatus of claim 4, wherein each of the recesses has a semi-circular profile.

6. The apparatus of claim 5, wherein the recesses are configured to communicate fluid along a longitudinal length of the inner shaft.

7. The apparatus of claim 6, wherein the outer shaft and the inflatable member are sized to fit within an airway of a patient, wherein the inflatable member is operably configured to dilate a stenosis in an airway of the patient upon inflation of the inflatable member.

8. The apparatus of claim 2, wherein the one or more features comprise a plurality of protrusions extending outwardly from the outer surface of the inner shaft along a longitudinal length of the inner shaft.

9. The apparatus of claim 8, wherein the plurality of protrusions comprises four protrusions oriented around the inner shaft.

10. The apparatus of claim 1, wherein the one or more features are formed in the outer shaft.

11. The apparatus of claim 10, wherein the one or more features comprise a plurality of protrusions extending inwardly from the inner surface of the outer shaft.

12. The apparatus of claim 11, wherein the plurality of protrusions comprises four protrusions.

13. The apparatus of claim 11, wherein each protrusion of the plurality of protrusions is semi-circular in shape, wherein each protrusion of the plurality of protrusions defines a lumen extending longitudinally through the outer shaft.

14. The apparatus of claim 13, wherein each lumen of each protrusion of the plurality of protrusions is in communication with the inflatable member.

15. The apparatus of claim 10, wherein the one or more features comprises at least one flat surface projecting from the inner surface of the outer shaft, wherein the at least one flat surface is integral with the inner surface of the outer shaft.

16. The apparatus of claim 15, wherein the at least one flat surface comprises a plurality of lumens extending along a longitudinal length of the outer shaft.

17. The apparatus of claim 16, wherein the plurality of lumens are in communication with the inflatable member.

18. The apparatus of claim 16, wherein at least one of the plurality of lumens is of a larger diameter than another lumen of the plurality of lumens.

19. A multi-lumen catheter assembly, comprising:
(a) an inner shaft, wherein the inner shaft comprises:
(i) an inner surface, wherein the inner surface defines a first lumen, and
(ii) an outer surface;
(b) an outer shaft coaxially disposed about the inner shaft, wherein the outer shaft is deformable inwardly toward the outer surface of the inner shaft, wherein the outer shaft comprises:
(i) an inner surface, and
(ii) an outer surface,
wherein the outer shaft is radially spaced from the inner shaft such that the inner surface of the outer shaft and the outer surface of the inner shaft together define a second lumen;
wherein one or both of the outer surface of the inner shaft or the inner surface of the outer shaft includes at least one longitudinally extending groove.

20. A multi-lumen catheter assembly, comprising:
(a) an inner shaft, wherein the inner shaft comprises:
(i) an inner surface, wherein the inner surface defines a first lumen, and
(ii) an outer surface;
(b) an outer shaft coaxially disposed about the inner shaft, wherein the outer shaft is deformable inwardly toward the outer surface of the inner shaft, wherein the outer shaft comprises:
(i) an inner surface, and
(ii) an outer surface,
wherein the outer shaft is radially spaced from the inner shaft such that the inner surface of the outer shaft and the outer surface of the inner shaft together define a second lumen;
wherein one or both of the outer surface of the inner shaft or the inner surface of the outer shaft includes at least one longitudinally extending protrusion configured to communicate fluid when the outer shaft is deformed inwardly toward the outer surface of the inner shaft.
